(19) United States
(12) Patent Application Publication
(10) Pub. No.: US 2009/0062511 A1
(43) Pub. Date: Mar. 5, 2009

(54) PROCESS FOR THE PREPARATION OF BIVALIRUDIN AND ITS PHARMACEUTICAL COMPOSITIONS

(76) Inventors: Raghavendracharyulu Venkata Palle, Hyderabad (IN); Sasikumar Pottayil Govindan Nair, Cochin (IN); Atul Kasturchand Godha, Ahmed Nagar (IN); Anant Madhavrao Marathe, Hyderabad (IN); Raghavendra Rao Morthala, Hyderabad (IN); Basanthi Devi, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/205,348

(22) Filed: Sep. 5, 2008

Related U.S. Application Data
(60) Provisional application No. 61/059,313, filed on Jun. 6, 2008.

(30) Foreign Application Priority Data
Sep. 5, 2007 (IN) 1983/CHE/2007

(51) Int. Cl. C07K 7/08

(52) U.S. Cl. 530/326

(57) ABSTRACT

The present application provides an improved process for the preparation of Bivalirudin and its pharmaceutical compositions.

The present application also provides an improved process for the purification of Bivalirudin.

### FILED OF THE APPLICATION

The present application relates to an improved process for the preparation of Bivalirudin and its pharmaceutical compositions.

The present application also relates to an improved process for the purification of Bivalirudin.

### BACKGROUND OF THE APPLICATION

Hirudin, a 65-amino acid polypeptide is a potent thrombin inhibitor naturally occurring in the salivary glands of medicinal leeches.

Bivalirudin, also known as hirulog-8, is a synthetic peptide based on hirudin and is a 20-amino acid polypeptide. It is chemically represented as D-phenylalanyl-L-prolyl-L-arginyl-L-prolyl-glycyl-glycyl-glycyl-glycyl-L -asparaginyl-glycyl-L-alpha-aspartyl-L-phenylalanyl-L-alpha-glutamyl-L-alpha-glutamyl-L-isoleucyl-L-prolyl-L-alpha-glutamyl-L-alpha-glutamyl-L -tyrosyl-L-leucine trifluoroacetate hydrate.

Bivalirudin directly inhibits thrombin, a key component in blood clot formation and extension. It is currently marketed in the US under the brand name Angiomax®.

The synthesis of peptides is generally carried out through the condensation of the carboxyl group of an amino acid, and the amino group of another amino acid, to form a peptide bond. A sequence can be constructed by repeating the condensation of individual amino acids in stepwise elongation, or, by condensation between two or more preformed peptide fragments. In both types of condensation, the amino and carboxyl groups that are not desired to participate in the reaction must be blocked/protected with protecting groups. In addition, reactive side chain functionalities of the amino acids also need to be protected.

In conventional solid phase peptide synthesis, the peptide-resin linkage is critical to the synthetic procedure. The linkage must be appropriately stable to deprotection of the amino blocking/protecting groups. If the linkage is not stable to deprotection conditions, the peptide will get prematurely cleaved from the resin. Further, the linkage may be readily cleavable upon completion of the synthesis of the peptide, preferably under conditions that will not damage the peptide being recovered. Hence, a balance between the resin peptide linkage retention during amino group deprotection and cleavage of completely synthesized peptide poses an opportunity of appropriate selection of resin, deprotecting agent, cocktail composition for cleavage of resin from peptide and global deprotection of linked amino acids in order to arrive on an improved process, wherein inherent process problems of peptide degradation, undesired impurities formations during the intermediate steps and industrial non-viability can be mitigated.

U.S. Pat. No. 5,196,404 describes a method for the preparation of Bivalirudin using BOC-L-Leucine-O-divinyl benzene resin. The process involves a sequential approach of adding Boc-protected amino acids on divinylbenzene resin. The peptide sequence obtained was fully deprotected and uncoupled from the resin using anhydrous HF: p-cresol: ethyl methyl sulfate, followed by Lyophilization. The crude obtained was purified by HPLC using a Vydac C-18 column to give pure peptide.

Chem. Pharm. Bull. 1996, 44, 1344-1350 describes the synthesis of various hirulog derivatives using conventional solution-phase methods. The process involves the use of Fmoc-protected amino acid p-alkoxy benzyl alcohol resin as the starting resin. The synthesis was performed using DCC or water soluble carbodiimide and HOBt as active ester coupling agents, and TFA in 1.5% water and 1.5% anisole as the cleavage solution.

European Journal of Biochemistry, 1999, 265, 598-605 discloses a process for the preparation of hirulog analogues (BZA-1 hirulog), which involves the use of:

(i) PyBop.DIEA in DMF for coupling of Fmoc-Glu-ODmab with Wang resin

(ii) use of 20% piperidine in DMF for Fmoc deprotection

(iii) reagent K (82.5% TFA, 5% Phenol, 5% thioanisole, 5% water, 2.5% 1,2-ethanedithiol) for cleavage of peptide from resin. However, it doesn't disclose the use of the above conditions for the preparation of Bivalirudin.

WO 2006/45503 A1 describes a process for the preparation of Bivalirudin, which involves the use of peptide resin conjugate comprising a 2-chloro-trityl handle, which required weakly acid conditions for cleavage of peptide from resin.

US 2007/0093423 A1 describes a process for preparing Bivalirudin peptide sequence on a hyper acid labile resin, which allow cleavage of the peptide from the resin in presence of mild acidic conditions, and involves the use of amino acids suitably protected with Boc or Fmoc. The US '423 application also disclose a process for the purification of Bivalirudin using a C18 RP-HPLC column in preparative HPLC.

Even though, the above mentioned prior art discloses diverse processes for the preparation of Bivalirudin, they are often not amenable to scale-up for preparing Bivalirudin.

Thus there still exists need for a more robust, cost effective and user convenient up-scalable process for the preparation of Bivalirudin.

### SUMMARY OF THE APPLICATION

The present application relates to an improved process for the preparation of Bivalirudin and its pharmaceutical compositions.

The present application also relates to an improved process for the purification of Bivalirudin.

In one aspect of the present application, it provides an improved process for the preparation of Bivalirudin, which comprises one or more of the steps of:
(1) Providing a capped resin comprising an anchored first protected amino acid;
(2) Selectively deprotecting the amino acid;
(3) Coupling the carboxyl terminus of the next N-protected amino acid to the amine from step 2);
(4) Repeating steps 2) and 3) to synthesize the desired peptide sequence; and
(5) Cleaving the peptide from the resin and isolating the peptide. In an embodiment of this aspect of the invention the resin provided in step (1) above is obtained by anchoring a first protected terminal amino acid to a resin followed by capping the resin as described herein. In still a further embodiment, the anchoring step employs MSNT&1-methylimidazole. In yet a further embodiment, capping occurs only after the anchoring step of the first protected amino acid.

In some embodiments, “providing” is accomplished by first anchoring a first protected amino acid to the resin followed by capping.

In accordance with some embodiments, cleavage of peptide from the resin not only involves cleavage of peptide but also involves Global deprotection (a process for deprotecting the protected amino acid in the peptide, which have additional functional groups) and is carried out by any of the two methods disclosed:
1. Using TFA/Phenol/Thioanisole/Water/Triisopropyplsilane (TIS) in a ratio of about 82.5%, 5%, 5%, 2.5%, and 5%
2. Using a cocktail mixture of reagents comprising of: TFA/Phenol/Water/TIS in the preferred proportions of about 76.5%/17.5%/4.3%/1.7% respectively

In another aspect of the present invention, the embodiment provides an improved process for the purification of Bivalirudin, which comprises one or more of the steps of:
1) Purification by neutral gradient method on Preparative High Performance Liquid Chromatography “HPLC” using PLRP-S column
2) Purification by acid gradient method on Preparative HPLC using PLRP-S column
3) Isolating Pure Bivalirudin.

In one of the particular aspect of the present application, the embodiment provides an improved process for the preparation of Bivalirudin, which comprises one or more of the steps of:
1) Providing a capped resin comprising an anchored first protected terminal amino acid. As noted above, the capped resin can be obtained by additional steps of anchoring a first protected amino acid and by capping a resin with anchored protected amino acid(s);
2) Selectively deprotecting the amino acid;
3) Coupling the carboxyl terminus of the next N-protected amino acid to the amine from step 3);
4) Repeating steps 3) and 4) to synthesize the desired peptide; and
5) Cleaving the peptide from the resin and global deprotection
6) Purifying Bivalirudin by using a PLRP-S column in preparative HPLC

The resin utilized in the process of the present invention for the preparation of Bivalirudin acts as support material and is selected from TentaGel TGA, TentaGel S PHB, TentaGel S AC, ChemMatrix Wang, Wang resin (1.2 mmol/g) or HMPB Chem Matrix. The selection of polymeric support and attached linker is very critical for overall outcome of the solid phase peptide synthesis. The ChemMatrix resin with Wang type linker used in one of the process of the present invention provides additional advantages over the other resins. The ChemMatrix support is made from highly stable ether bonds and has superior mechanical and swelling properties. Resins like Tentagel are also found be very effective for the preparation of Bivalirudin and are comprising of grafted copolymers consisting of a low cross linked polystyrene on which polyethylene glycol is grafted.

In a further another embodiment, the present invention provides pharmaceutical compositions comprising a therapeutically effective amount of Bivalirudin or a pharmaceutically acceptable salts, its pharmaceutically acceptable analogs, polymorphs, solvates, or mixtures thereof, and optionally, one or more pharmaceutically acceptable excipients.

In another embodiment, the present invention provides processes for preparing pharmaceutical compositions containing a therapeutically effective amount of Bivalirudin or a pharmaceutically acceptable salts, its pharmaceutically acceptable analogs, polymorphs, solvates, or mixtures thereof, and optionally one or more pharmaceutically acceptable excipients.

In still another embodiment, there is provided a process for the preparation of Bivalirudin comprising one or more of the steps of:
1) Anchoring a first protected terminal amino acid to a resin and in a further embodiment, this is accomplished using MSNT and 1-methylimidazole;
2) Capping of resin obtained after step-1, and, in a further embodiment this is accomplished using anhydride of acetic acid or otherwise providing a first protected terminal amino acid on a capped resin;
3) Selectively deprotecting the amino acid using nucleophilic base;
4) Coupling the carboxyl terminus of the next N-protected amino acid to the amine from step 3) in presence of DIC and HOBt;
5) Repeating steps 3) and 4) to synthesize the desired peptide sequence;
6) Cleaving the peptide from the resin and global deprotection using cocktail mixture comprising a composition of TFA/Phenol/Water/TIPS to obtain crude Bivalirudin and, optionally
7) Purifying crude Bivalirudin by using a PLRP-S column in preparative HPLC by neutral gradient followed by acid gradient method.

In yet another embodiment, there is provided a process for preparation of Bivalirudin anchored to polymeric resin comprising one or more of the steps of:
a) Preparation of Fmoc-Leu-Resin by reaction of Wang Resin with Fmoc-Leu-OH in Presence of MSNT, dichloromethane, tetrahydrofuran and 1-Methyl imidazole.
b) Capping of the resin anchored with Fmoc-Leu using a capping solution comprising of acetic anhydride, pyridine, and dichloromethane in the ratio of about 1:8:8 respectively.
c) Preparation of Fmoc-Tyr(tBu)-Leu-Resin after deprotection of Fmoc group using deprotection reagent (20% PIP in DMF) followed by reacting with Fmoc-Tyr(tBu)-OH in presence of HOBt and DIC.
d) Preparation of Fmoc-Glu(OtBu)-Tyr(tBu)-Leu-Resin after deprotection of Fmoc group using deprotection reagent (20% PIP in DMF) followed by reacting with Fmoc-Glu(OtBu)-OH, in presence of HOBt and DIC.
e) Preparation of Fmoc-Glu(OtBu)-Glu(OtBu)-Tyr(tBu)-Leu-Resin after deprotection of Fmoc group using deprotection reagent (20% PIP in DMF) followed by reacting with Fmoc-Glu(OtBu)-OH, in presence of HOBt and DIC.
f) Preparation of Fmoc-Pro-Glu(OtBu)-Glu(OtBu)-Tyr(tBu)-Leu-Resin after deprotection of Fmoc group using deprotection reagent (20% PIP in DMF) followed by reacting with Fmoc-Pro-OH, in presence of HOBt and DIC.
g) Preparation of Fmoc-Ile-Pro-Glu(OtBu)-Glu(OtBu)-Tyr(tBu)-Leu-Resin after deprotection of Fmoc group using deprotection reagent (20% PIP in DMF) followed by reacting with Fmoc-Ile-OH, in presence of HOBt and DIC. h) Preparation of Fmoc-Glu(OtBu)-Ile-Pro-Glu(OtBu)-Glu(OtBu)-Tyr(tBu)-Leu-Resin after deprotection of Fmoc group using deprotection reagent (20% PIP in DMF) followed by reacting with Fmoc-Glu(OtBu)-OH, in presence of HOBt and DIC.
i) Preparation of Fmoc-Glu(OtBu)-Glu(OtBu)-Ile-Pro-Glu(OtBu)-Glu(OtBu)-Tyr(tBu)-Leu-Resin after deprotection of Fmoc group using deprotection reagent (20% PIP in DMF) followed by reacting with Fmoc-Glu(OtBu)-OH, in presence of HOBt and DIC.
j) Preparation of Fmoc-Phe-Glu(OtBu)-Glu(OtBu)-Ile-Pro-Glu(OtBu)-Glu(OtBu)-Tyr(tBu)-Leu-Resin after deprotection of Fmoc group using deprotection reagent (20% PIP in DMF) followed by reacting with Fmoc-Phe-OH, in presence of HOBt and DIC.
k) Preparation of Fmoc-Asp(OtBu)-Phe-Glu(OtBu)-Glu(OtBu)-Ile-Pro-Glu(OtBu)-Glu(OtBu)-Tyr(tBu)-Leu-Resin after deprotection of Fmoc group using deprotection reagent (40% PIP in DMF) followed by reacting with Fmoc-Asp(OtBu)-OH, in presence of HOBt and DIC.
l) Preparation of Fmoc-Gly-Asp(OtBu)-Phe-Glu(OtBu)-Glu(OtBu)-Ile-Pro-Glu(OtBu)-Glu(OtBu)-Tyr(tBu)-Leu-Resin after deprotection of Fmoc group using deprotection reagent (20% PIP in DMF) followed by reacting with Fmoc-Gly-OH, in presence of HOBt and DIC.
m) Preparation of Fmoc-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin after deprotection of Fmoc group using deprotection reagent (20% PIP in DMF) followed by reacting with Fmoc-Asn(trt)-OH, in presence of HOBt and DIC.
n) Preparation of Fmoc-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin after deprotection of Fmoc group using deprotection reagent (20% PIP in DMF) followed by reacting with Fmoc-Gly-OH, in presence of HOBt and DIC.
o) Preparation of Fmoc-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin after deprotection of Fmoc group using deprotection reagent (20% PIP in DMF) followed by reacting with Fmoc-Gly-OH, in presence of HOBt and DIC.
p) Preparation of Fmoc-Gly-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin after deprotection of Fmoc group using deprotection reagent (20% PIP in DMF) followed by reacting with Fmoc-Gly-OH, in presence of HOBt and DIC.
q) Preparation of Fmoc-Gly-Gly-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin after deprotection of Fmoc group using deprotection reagent (20% PIP in DMF) followed by reacting with Fmoc-Gly-OH, in presence of HOBt and DIC.
r) Preparation of Fmoc-Pro-Gly-Gly-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin after deprotection of Fmoc group using deprotection reagent (20% PIP in DMF) followed by reacting with Fmoc-Pro-OH, in presence of HOBt and DIC.
s) Preparation of Fmoc-Arg(Pbf)-Pro-Gly-Gly-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu) -Leu-Resin after deprotection of Fmoc group using deprotection reagent (20% PIP in DMF) followed by reacting with Fmoc-Arg(Pbf)-OH, in presence of HOBt and DIC.
t) Preparation of Fmoc-Pro-Arg(Pbf)-Pro-Gly-Gly-Gly-Gly-Asn(trt)-Gly-Asp(OtBu)-Phe-Glu(OtBu)-Glu(OtBu)-Ile-Pro-Glu(OtBu)-Glu(OtBu) -Tyr(tBu)-Leu-Resin after deprotection of Fmoc group using deprotection reagent (20% PIP in DMF) followed by reacting with Fmoc-Pro-OH, in presence of HOBt and DIC.
u) Preparation of Boc-D-Phe-Pro-Arg(Pbf)-Pro-Gly-Gly-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin after deprotection of Fmoc group using deprotection reagent (20% PIP in DMF) followed by reacting with Boc-D-Phe-OH, in presence of HOBt and DIC.
v) Washed the resin with methanol, followed by methyl tert butyl ether.
w) Drying the resin anchored with protected Bivalirudin under vacuum.
x) Cleaving the peptide from the resin and global deprotecting using cocktail mixture comprising a composition of TFA/Phenol/Water/TIS in dichloromethane solvent to provide Bivalirudin.

### DETAILED DESCRIPTION OF THE APPLICATION

While the specification concludes with the claims particularly pointing and distinctly claiming the invention, it is believed that the present invention will be better understood from the following description. All percentages and ratios used herein are by weight of the total composition and all measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Note that while the specification and claims may refer to a final product such as, for example, a tablet or other dosage form of the invention as, for example, containing particles having a certain particle size or distribution, or a certain type of, for example, a specific form of a filler, it may be difficult to tell from the final dosage form that the recitation is satisfied. However, such a recitation may be satisfied if the materials used prior to final production (in the case of a tablet for example, blending and tablet formulation), for example, meet that recitation. Indeed, as to any property or characteristic of a final product which cannot be ascertained from the dosage form directly, it is sufficient if that property resides in the components recited just prior to final production steps.

A reference to a molecule such as, in this case, Bivalirudin, unless otherwise specified or inconsistent with the disclosure in general, refers to any salt, crystalline or amorphous form, optical isomer and/or solvate form thereof.

When a molecule or other material is identified herein as “pure”, it generally means, unless specified otherwise, that the material is about 99% pure or more. In general, this refers to purity with regard to unwanted residual solvents, reaction byproducts, impurities and unreacted starting materials. In the case of stereoisomers or polymorphs, “pure” also means 99% of one enantiomer or diastereomer, as appropriate or one polymorph. “Substantially” pure means, the same as “pure” except that the lower limit is about 98% pure or more and likewise, “essentially” pure means the same as “pure” except that the lower limit is about 95% pure.

The present application relates to an improved process for the preparation of Bivalirudin and its pharmaceutical compositions.

The present application also relates to an improved process for the purification of Bivalirudin.

In one aspect of the present application, it provides an improved process for the preparation of Bivalirudin, which comprises the steps of:
(1) Anchoring the first protected terminal amino acid to a resin, followed by capping of the resin;
(2) Selectively deprotecting the amino acid;
(3) Coupling the carboxyl terminus of the next N-protected amino acid to the amine from step 2);
(4) Repeating steps 2) and 3) to synthesize the desired peptide sequence; and
(5) Cleaving the peptide from the resin and isolating the peptide
   All of the steps for this process are individually described herein below.

Step 1): Anchoring the First Protected Terminal Amino Acid to a Resin, Followed by Capping of Resin;

The process of Step (1) involves anchoring of the first protected terminal amino acid to a resin, followed by capping of resin. The resin utilized in this step of the process of the present invention for the preparation of Bivalirudin acts as support material and is selected from TentaGel TGA, TentaGel S PHB, TentaGel S AC, ChemMatrix Wang, Wang resin (1.2 mmol/g) or HMPB Chem Matrix. The selection of polymeric support and attached linker is very critical for overall outcome of the solid phase peptide synthesis. The ChemMatrix resin with Wang type linker used in one of the process of the present invention provides additional advantages over the other resins. The ChemMatrix support is made from highly stable ether bonds and has superior mechanical and swelling properties. Resins like TentaGel are also found be very effective for the preparation of Bivalirudin and are comprising of grafted copolymers consisting of a low cross linked polystyrene on which polyethylene glycol is grafted.

In a preferred embodiment TentaGel S PHB or Wang resin are used in the process of the present invention.

Synthesis can be carried out automatically, for example using an automated peptide synthesizer, using either the synthesis programs i.e. programmed synthesis provided by the manufacturer or those constructed or designed by the users. The overall process may be carried out in an inert atmosphere, i.e. Nitrogen or Argon or the like.

The first amino acid anchored to the resin is typically L-leucine, wherein the amino terminus of L-leucine is blocked by a protecting group.

Alternatively the resin may be purchased pre-loaded with L-leucine, which may either have a free N-terminus or N-terminus protected with a protecting group. Whether purchased or made, these capped resins with anchored protected terminal amino acids are “provided,” a term which here covers the materials from any origin.

Suitable protecting groups for blocking the amino terminus include but not limited to 9-fluorenyl-methyloxycarbonyl (Fmoc), 2-(4-biphenylyl)-2-propyloxycarbonyl (Bpoc), propargyloxycarbonyl (Poc) and t-butyloxycarbonyl (Boc), etc.

In a preferred embodiment Fmoc is used as the protecting group for blocking the amino terminus and the protected amino acid is Fmoc-Leu-OH.

The resin is suspended in an organic solvent, which swells the resin. The organic solvent utilized for swelling or soaking the resin may be selected from methylene chloride, tetrahydrofuran, N,N-dimethylformamide or N-methylpyrrolidone. The process can optionally be repeated with the solvent system selected. The resin is then treated with N-terminus protected amino acid in the presence of an organic coupling agent for a desired period of time to affect the coupling.

The amount of protected amino acid used in the anchoring step is normally in excess molar quantities and can range from about 1M to about 12M with respect to the resin loading capacity, preferably 4-7 moles of the Fmoc-Leu-OH is used.

The coupling agent that can be used in the anchoring step may be selected from a combination of DIC & HOBt, DCC & HOBt, DCC & DMAP, PPh₃ & DEAD or MSNT & N-methylimidazole.

The amount of individual coupling agents in the combination of coupling agent selected from the above, may range from about 1 to about 8 molar equivalents with respect to one molar equivalent of resin used with respect to resin loading capacity.

In one of the preferred embodiment, about 6 molar equivalents of MSNT and about 3.75 molar equivalents of N-methyl imidazole per molar resin was used as the coupling agent.

Optionally, the coupling of amino acid with preferred molar equivalents may also be carried out in two steps to increase the coupling efficiency, wherein the coupling reagent or protected amino acid or both may be utilized in two or more lots.

The coupling reaction may be carried out in a suitable solvent. The term “suitable solvent” refers to any solvent, or mixture of solvents, that afford a medium within which the desired reaction is carried out. The solvents that may be used in the coupling step include but are not limited to dichloromethane, tetrahydrofuran, dimethylformamide, N-methylpyrrolidone or the mixtures thereof.

The temperature at which the coupling is carried out may range from about 15° C. to about 40° C.

After the completion of the reaction, the resin may be optionally washed with solvents such as dichloromethane, dimethylformamide to remove residual reagents and byproducts. The process may be repeated, if desired.

Before proceeding for the next steps after anchoring the first protected terminal amino acid, the unreacted linkers on the resin (polymer) are desired to be appropriately protected in order to avoid the undesired peptide chain formation. Preferably, the free functional groups on the polymeric resin may be protected as their ester. This process is referred to as Capping and may be carried out after anchoring the first protected amino acid to the resin.

In an embodiment, the capping may be carried out by using acetic anhydride in the presence of pyridine and a suitable solvent. Preferably, dichloromethane is used as suitable solvent for capping.

In a preferred embodiment the capping solution may comprise of acetic anhydride, pyridine and dichloromethane in the preferred ratio of about 1:8:8 (v/v)

Optionally, capping may also be repeated at some or even at each stage of the synthesis directly after the coupling to block unreacted functional groups.

Step-(2) Selectively Deprotecting the Amino Acid

The protected amino acid anchored to the resin may be selectively deprotected by method known in the art, for example, using an appropriate nucleophilic base such as 20% piperidine in suitable solvent like dimethylformamide (“DMF”). Optionally the process of selective deprotection may also be repeated.

In a preferred embodiment, Fmoc-L-leucine i.e. Fmoc protected amino acid anchored to Wang resin obtained from step 1) or commercially may be selectively deprotected by using about 15% to about 50% piperidine in DMF (v/v), preferably 20% piperidine in DMF (v/v). A preferred concentration of nucleophilic base with respect to resin may range from about 8% to about 12% w/v.

The process of selectively deprotection of protected amino acid attached to the resin or in the peptide chain may be carried out at a temperature in the range of 5° to 30° C. Preferred temperature for the selective deprotection is 20-25° C., however, it may vary from amino acid to amino acid during sequential protected amino acid addition. For example, this temperature for amino acid like Phenyl alanine may vary from about 6° to about 10° C.

The process of selective deprotection further comprises washing the deprotected amino acid with a suitable solvent such as dichloromethane or dimethylformamide or their mixture to remove residual reagents and byproducts.

The coupling efficiency after each coupling step may be monitored during synthesis by means of a Kaiser test or any other suitable test. The individual coupling steps, if showing unexpectedly low coupling efficiency may also be repeated prior to proceeding for deprotection and coupling with next amino acid of the sequence.

Coupling the carboxyl terminus of the next N-protected amino acid to the amine from the selective deprotection step.

Coupling the carboxyl terminus of the next N-protected amino acid to the amine from the selective deprotection step involves coupling with next N-protected amino acid of the sequence to the deprotected amino acid anchored on the resin. The next amino acid for Bivalirudin in the sequence is L-tyrosine, which is coupled after protection or using directly protected L-tyrosine with the free amine terminus of L-leucine-Resin obtained from the selective deprotection step in presence of a coupling agent.

In an embodiment of the present invention, protected L-tyrosine used is Fmoc-Tyr(tBu)-OH.

The amount of protected amino acid used in the coupling step is normally in excess molar quantities and may range from about 1 to about 7 molar equivalents, per molar equivalent of resin with respect to resin loading capacity. Preferably, about 2 to about 4 molar equivalent of the protected amino acid is utilized for most of the amino acids, however, the molar equivalent of the protected amino acid utilized may further vary for some of the amino acids like Tyr, Glu, Pro, Ileu, Phe, Asn, and Gly and preferably used in the range of about 1.5 to about 2.5 molar equivalent. For amino acids like Arg, the molar ratio may be used in the range of about 5.5 to about 6.5 molar equivalent.

Suitable coupling agents include, but are not limited to TBTU, DCC, DIC, HBTU, BOP, PyBOP, PyBrOP, PyCIOP, TCTU, EEDQ and IIDQ or preformed active esters, MSNT, N-methylimidazole either individually or as a combination thereof. The amount of individual coupling agents used may range from about 1 to about 6 molar equivalents, per molar equivalent of resin with respect to resin loading capacity. Preferably, 3 molar equivalents of individual coupling agents per molar equivalent of the resin with respect to resin loading capacity may be used.

In one of the preferred embodiment of the present invention, about 3 molar equivalents each of DIC and HOBt may be used as the coupling agent.

The coupling reaction may be carried out in a suitable solvent. The term “suitable solvent” refers to any solvent, or mixture of solvents, that afford a medium within which the desired reaction is carried out. The solvents that may be used in the coupling step include but are not limited to dichloromethane, tetrahydrofuran, dimethylformamide, N-methylpyrrolidone or the mixtures thereof.

The temperature at which the coupling is carried out may range from about 15° C. to about 40° C. The overall process may be carried out in an inert atmosphere, i.e. Nitrogen or Argon.

After the completion of the reaction, the resin may be optionally washed with solvents such as dichloromethane, dimethylformamide to remove residual reagents and byproducts. The process may be repeated, if desired.

The deprotecting and coupling steps just described are repeated as needed to synthesize the desired peptide sequence.

This step involves repeating the prior two steps to synthesize the desired peptide sequence, which includes coupling and deprotecting of subsequent protected (preferably Fmoc protected) amino acids i.e. Glu, Glu, Pro, Ileu, Glu, Glu, Phe, Asp, Gly, Asn, Gly, Gly, Gly, Gly, Pro, Arg, Pro and D-Phe.

The steps of the deprotecting the Fmoc group of the amino acid and coupling the next suitably protected amino acid of the sequence may be carried according to the process described previously to get desired Bivalirudin peptide sequence.

The functional group present on the amino acids used in the process of the present invention may be appropriately protected to avoid any undesired side reaction products. Suitable protective groups are described in the Literature (see, for example, P Wuts and T. W. Greene, “Protective Groups in Organic Synthesis”, John Wiley & Sons, 4^{th} edition, 2007). The protecting group may vary depending upon the particular amino acid which may include, but are not limited to Boc, Pbf, tBu and Trt.

The Fmoc protected amino acids are commercially available or may be sourced from Adv. Chem. Tech. or may be prepared according to procedures given in the literature. The order by which the protected amino acids are added up to synthesize Bivalirudin peptide sequence comprises of Fmoc-Glu(Otbu)-OH, Fmoc-Glu(Otbu)-OH, Fmoc-Pro-OH, Fmoc-Ileu-OH, Fmoc-Glu(Otbu)-OH, Fmoc-Glu(Otbu)-OH, Fmoc-Phe-OH, Fmoc-Asp(Otbu)-OH, Fmoc-Gly-OH, Fmoc-Asn(trt)-OH, Fmoc-Gly-OH, Fmoc-Gly-OH, Fmoc-Gly-OH, Fmoc-Gly-OH, Fmoc-Pro-OH, Fmoc-Arg(pbf)-OH, Fmoc-Pro-OH, and Boc-D-Phe-OH.

The next step is cleaving the peptide from the resin and isolating the peptide. This step involves cleaving the peptide from the resin to isolate the desired peptide.

The cleavage of the peptide from the solid support may be accomplished by any conventional method. The process may also result in both cleaving the peptide from the resin and global deprotection of the side chain protecting group of the amino acids to provide Bivalirudin. The overall process may be carried out in an inert atmosphere, i.e. Nitrogen or Argon.

In an embodiment, the process of the present invention involves cleaving and global deprotection of Boc-D-Phe-Pro-Arg(Pbf)-Pro-Gly-Gly-Gly-Gly-Asn(Trt)-Gly-Asp(OtBu-)-Phe-Glu(OtBu)-Glu(OtBu)-Ile-Pro-Glu(OtBu)-Glu(OtBu)-Tyr(tBu)-Leu-Resin to give crude Bivalirudin.

The step of cleaving the peptide from the resin involves treating the protected peptide anchored to the resin with an acid and at least one scavenger.

The peptide cleavage reagent used in the process of the present invention is a cocktail mixture of acid, scavengers and solvents.

The acid utilized in the cleavage reagents may be selected from trifluoroacetic acid (TFA), difluoroacetic acid or monofluoroacetic acid.

Scavengers such as EDT, DDM, TES, TIS, phenol, thioanisole and water or in any combination thereof may be used in the process of the present invention.

Preferably, a cocktail mixture comprising TFA/Phenol/Thioanisole/Water/Triisopropyplsilane (TIS) in a ratio of about 82.5%, 5%, 5%, 2.5%, and 5% respectively may be used as the peptide cleaving and global deprotecting reagent.

More preferably, a cocktail mixture comprising TFA/Phenol/Water/TIS in a ratio of about 76.5%, 17.5%, 4.3%, and 1.7% respectively may also be used as the peptide cleaving and global deprotecting reagent.

The solvent used in this cleaving step of the process of the present invention may be selected from but not limited to dichloromethane, trichloromethane or the like. Dichloromethane is used as preferred solvent. The solvent may also help in swelling the resin prior to effective cleavage of the peptide.

The amount of TFA used for the purpose of cleavage of peptide from resin and global deprotection in the cocktail mixture may range from 60% to 90% with respect to its concentration in DCM. Even though the TFA concentration utilized is higher for the cleavage of peptide from resin and global deprotection, however, inventors of the present application observed that such composition of TFA resulted in desired cleavage and global deprotection without affecting the peptide yield.

The temperature at which the cleavage and global deprotection may be carried out ranging from about 15° C. to about 40° C. Preferred temperature for cleavage and global deprotection may be in the range of about 25°-30° C.

After the completion of the reaction, the reaction mixture may optionally be filtered and washed with acid or an organic solvent. Crude Bivalirudin may be isolated by combining the reaction mass with an organic solvent, preferably by combining with an ether solvent.

Ether solvents that may be used include but are not limited to diethyl ether, diisopropyl ether, tert-butyl methyl ether, tert-butyl ethyl ether, tert-amyl methyl ether, isopropyl ether and the like or combinations thereof.

The isolation may be carried out by adding an ether solvent to the reaction mass or by adding reaction mass to the ether solvent selected. Preferably, the reaction mass is added to an ether solvent. More preferably, the reaction mass is added to an ether solvent precooled to a temperature of about −5° C. to about 5° C.

The obtained suspension may be maintained at a temperature of about 0° C. to about 15° C., preferably at a temperature of about 0° C. to about 5° C. to effect the complete precipitation of the product.

The obtained precipitate may be separated using conventional techniques known in the art. One skilled in the art may appreciate that there are many ways to separate a solid from the mixture, for example it can be separated by using techniques such as filtration by gravity or by suction, centrifugation, decantation, and the like. The obtained crude product may be optionally washed with an organic solvents preferably ether and subjected to drying under continuous nitrogen purging. The isolated product—Bivalirudin may optionally be further purified, if desired.

In another aspect of the present invention, the embodiment provides an improved process for the purification of Bivalirudin, which comprises the steps of:
1) Purification by neutral gradient method on Preparative HPLC using PLRP-S column
2) Purification by acid gradient method on Preparative HPLC using PLRP-S column
3) Isolating pure Bivalirudin.
   The purification process of Bivalirudin is carried out on preparative HPLC, wherein often a C-18 or C-8 column is utilized on reversed phase. The process of the present invention utilizes a preparative HPLC column, preferably PLRP-S, which is not a C-18 or C-8 column unlike the conventional practices to use such columns for preparative chromatography. Such purification resulted in the preparation of highly pure Bivalirudin.

All of the steps for the purification process are individually described herein below.

### 1) Purification by Neutral Gradient Method on Preparative HPLC Using PLRP-S Column

Purification for crude Bivalirudin is carried out firstly in a neutral gradient medium, which comprises dissolution of crude peptide in ammonium acetate buffer and loading onto the column, which is packed with PLRP-S stationary phase. The stationary phase is a matrix of polystyrene-divinyl benzene co-polymer. The material is then eluted with a gradient of ammonium acetate (Buffer A) and methanol/acetonitrile (Buffer B) on a column.

Preferably the gradient of ammonium acetate (Buffer A) may have concentration of about 40 mM and methanol/acetonitrile (Buffer B) volumes may range from 50-75/50-25.

During elution, fractions are collected at regular intervals using a PREP LC system. The collected fractions are assayed by HPLC to determine the purity and fraction with desired purities may be pooled together for further purification.

### 2) Purification by Acid Gradient Method on Preparative HPLC Using PLRP-S Column

The pooled fraction obtained from the previous purification step using neutral gradient is subjected to further purification separately using the same column by elution with a gradient comprising of buffer A: Orthophosphoric acid (adjusted to pH ˜3.0 with Triethylamine) and B: Acetonitrile.

In one of the preferred embodiment, the concentration of Orthophosphoric acid used as Buffer A may be about 0.3% to about 1% in water.

During the elution, the desired pure fractions are collected again and assayed by HPLC.

### 3) Isolating Pure Bivalirudin

The purified Bivalirudin pooled fraction from step-2) is then subjected to desolvatization to remove acetonitrile solvent. The desolvated concentrated pure pool is then loaded onto the same PLRP-S column for desalting step.

Desalting step is carried out by continuous washing of the loading column till the pH of the wash eluent increases to about ˜5.0. Preferably such washing with water may be repeated 3-4 times in order to get the complete salt removal.

Further, the column eluted with four volumes of 0.1% TFA in water followed by elution with a gradient of 0.1% TFA in water and acetonitrile to collect the TFA salt of

Bivalirudin fractions and analyzed by HPLC assay method to ascertain the purity of fractions. Fractions with desired purity preferably greater than 96.5% purity may be considered as pure fractions.

The pure pooled fraction so obtained may be subjected to Lyophilization under the set parameters of Lyophilization to collect the lyophilized powder which may assayed by purity method of HPLC to ensure that it meets API specifications.

Bivalirudin obtained by the process of the present invention was analyzed for purity by HPLC. HPLC measurements of Bivalirudin samples for chemical purity were performed using Waters system, equipped with CRONUSIL-M C18, 250×4.6 mm, 3 μm or equivalent column. The column is maintained at a temperature ranging from about 30° C. to about 40° C. and the sample temperature may range from about 8° C. to about 12° C. and a UV detector operated on 210 nm. Analyses were performed using the following mobile phase, at flow rate of about 0.8 ml/minute and a run time 130 minutes.

Mobile phase A: Dissolve 0.5 g of sodium 1-butanesulphonate in 100 ml of Milli Q water, add 3 ml Orthophosphoric acid to the above solution and adjust the pH to 3.0±0.05 with trimethylamine. Filtered through 0.45 μm membrane filter.

Mobile phase B: A mixture of methanol and acetonitrile in the ratio of 750:250 and filtered through 0.45 μm membrane filter.

Elution: was carried out as per the user specific Gradient program

The HPLC chromatogram obtained by the above analytical method of the present invention revealed that Bivalirudin contains impurities at relative retention time (“RRT”) of about 0.928, 0.967 and 1.030 having content less than 1% and preferably less than 0.5%. The above mentioned impurities correspond to D-Phe¹², Pentagly, and Trigly respectively. These impurities are homologous impurities formed during Bivalirudin preparation process and elute more often adjacent to the Bivalirudin peak. Such elution of these impurities by the HPLC method of the presence invention provides inventive merit in resolving and detecting the presence of these impurities. The HPLC method of the present invention is also robust enough to separate the unknown impurities at a level of less than 0.2%, which are detected at RRT of about 0.601, 0.894, 0.934, and 1.090.

In one of the particular aspect of the present application, the embodiment provides an improved process for the preparation of Bivalirudin, which comprises the steps of:
1) Providing a first protected terminal amino acid on a capped resin (by anchorage and capping or purchase);
2) Selectively deprotecting the amino acid;
3) Coupling the carboxyl terminus of the next N-protected amino acid to the amine from step 2);
4) Repeating steps 2) and 3) to synthesize the desired peptide; and
5) Cleaving the peptide from the resin and global deprotection
6) Purifying Bivalirudin by using a PLRP-S column in preparative HPLC
   All of the steps for this process are individually described herein below.

Step 1): Anchoring the First Protected Terminal Amino Acid to a Resin

The process of Step (1) involves anchoring of the first protected terminal amino acid to a resin.

The first protected amino acid used is Fmoc-Leu-OH and the resin used in the process may be selected from Tentagel S PHB or Wang resin (1.2 mmol/g). The overall process may be carried out in an inert atmosphere, i.e. Nitrogen or Argon or the like.

The resin is suspended in an organic solvent which swells the resin and may be selected from methylene chloride, tetrahydrofuran, N,N-dimethylformamide or N-methylpyrrolidone. The process can optionally be repeated with the solvent system selected.

The resin is then treated with N-terminus protected amino acid in the presence of an organic coupling agent for a desired period of time to affect the coupling

The amount of protected amino acid used in step 1) is normally in excess molar quantities and can range from about 1M to about 8M equivalents, per molar equivalent of resin utilized with respect to resin loading capacity. Preferably 6 molar equivalents of the Fmoc-Leu-OH is used.

The coupling agent that can be used in step 1) may be selected from a combination of DIC & HOBt, or MSNT & N-methylimidazole.

In an embodiment, about 6 molar equivalents of MSNT and about 3.75 molar equivalents of N-methyl imidazole per molar resin was used with respect to resin loading capacity as the coupling agent.

Optionally, the coupling of amino acid with preferred molar equivalents may also be carried out in two or more steps to increase the coupling efficiency, wherein the coupling reagent or protected amino acid or both may be utilized in two or more lots.

The coupling reaction may be carried out in a suitable solvent. The solvents that may be used in the coupling step include but are not limited to dichloromethane, tetrahydrofuran, dimethylformamide, N-methylpyrrolidone or the mixtures thereof.

The temperature at which the coupling is carried out may range from about 15° C. to about 40° C.

After the completion of the reaction, the resin may be optionally washed with solvents such as dichloromethane, dimethylformamide to remove residual reagents and byproducts. The process may be repeated, if desired.

The capping of unreacted linkers on the resin (polymer) are desired to be appropriately protected and may be carried out by using acetic anhydride in the presence of pyridine and a suitable solvent. Preferably, dichloromethane is used as suitable solvent for capping.

In a preferred embodiment the capping solution may comprise of acetic anhydride, pyridine and dichloromethane in the preferred ratio of about 1:8:8 (v/v)

Optionally, capping may also be repeated at each stage of the synthesis directly after the coupling to block unreacted functional groups.

Step-(2) Selectively Deprotecting the Amino Acid;

The protected amino acid anchored to the resin may be selectively deprotected by method known in the art, for example, using an appropriate nucleophilic base such as 20% piperidine in suitable solvent like DMF. Optionally the process of selective deprotection may also be repeated.

In a preferred embodiment, Fmoc-L-leucine i.e. Fmoc protected amino acid anchored to Wang resin obtained from step 1) may be selectively deprotected by using about 20% piperidine in DMF (v/v). A preferred concentration of nucleophilic base with respect to resin may range from about 8% to about 12% w/v.

The process of step 2) i.e. Selectively deprotection of protected amino acid attached to the resin or in the peptide chain may be carried out at a temperature in the range of 0 to 30° C.

The process of step 2) further comprises washing the deprotected amino acid with a suitable solvent such as dichloromethane or dimethylformamide or their mixture to remove residual reagents and byproducts.

Step-3) Coupling the Carboxyl Terminus of the Next N-Protected Amino Acid to the Amine from Step 2);

The next amino acid for Bivalirudin in the sequence is L-tyrosine, wherein Fmoc-Tyr(tBu)-OH is coupled with the free amine terminus of L-leucine-Resin obtained from step (3) in presence of a coupling agent.

The amount of protected amino acid used in step (2) is normally in excess molar quantities and may range from about 1 to about 7 molar equivalents, per molar equivalent of resin with respect to resin loading capacity. Preferably, about 2 to about 4 molar equivalent of the protected amino acid are utilized.

Suitable coupling agents include, but are not limited to TBTU, DCC, DIC, HBTU, HOBt, BOP, PyBOP, PyBrOP, PyCIOP, TCTU, EEDQ and IIDQ or preformed active esters, MSNT, N-methylimidazole either individually or as a combination thereof. The amount of individual coupling agents used may range from about 1 to about 6 molar equivalents, per molar equivalent of resin.

In one of the preferred embodiment of the present invention, about 3 molar equivalents each of DIC and HOBt may be used as the coupling agent.

The coupling reaction may be carried out in a suitable solvent. The solvents that may be used in the coupling step include but are not limited to dichloromethane, tetrahydrofuran, dimethylformamide, N-methylpyrrolidone or the mixtures thereof.

The temperature at which the coupling is carried out may range from about 15° C. to about 40° C. The overall process may be carried out in an inert atmosphere, i.e. Nitrogen or Argon.

After the completion of the reaction, the resin may be optionally washed with solvents such as dichloromethane, dimethylformamide to remove residual reagents and byproducts. The process may be repeated, if desired.

Step-(4) Repeating Steps 2) and 3) to Synthesize the Desired Peptide;

Step-5 involves repeating steps (2) and (3) to synthesize the desired peptide sequence, which includes coupling and deprotecting of subsequent protected amino acids, wherein the protected amino acids are added up to synthesize Bivalirudin peptide in the sequence of Fmoc-Glu(Otbu)-OH, Fmoc-Glu(Otbu)-OH, Fmoc-Pro-OH, Fmoc-Ileu-OH, Fmoc-Glu(Otbu)-OH, Fmoc-Glu(Otbu)-OH, Fmoc-Phe-OH, Fmoc-Asp(Otbu)-OH, Fmoc-Gly-OH, Fmoc-Asn(trt)-OH, Fmoc-Gly-OH, Fmoc-Gly-OH, Fmoc-Gly-OH, Fmoc-Gly-OH, Fmoc-Pro-OH, Fmoc-Arg(pbf)-OH, Fmoc-Pro-OH, and Boc-D-Phe-OH.

The molar equivalent of the protected amino acid utilized may further vary for some of the amino acids like Tyr, Glu, Pro, Ileu, Phe, Asn, and Gly and preferably used in the range of about 1.5 to about 2.5 molar equivalent. For amino acids like Arg, the molar ratio may be used in the range of about 5.5 to about 6.5 molar equivalents.

The steps of the deprotecting the Fmoc group of the amino acid and coupling the next suitably protected amino acid of the sequence may be carried according to the process described in step (2) and step (3) respectively to get desired Bivalirudin peptide sequence.

Step (5) Cleaving the Peptide from the Resin and Global Deprotection

The cleavage of the peptide from the solid support may result in both cleaving the peptide from the resin and global deprotection of the side chain protecting group of the amino acids to provide Bivalirudin. The overall process may be carried out in an inert atmosphere, i.e. Nitrogen or Argon.

In an embodiment, the process of the present invention involves cleaving and global deprotection of Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin to give crude Bivalirudin.

The step of cleaving the peptide from the resin involves treating the protected peptide anchored to the resin with an acid, at least one scavenger and in the presence of solvent.

Preferably, a cocktail mixture comprising TFA/Phenol/Water/TIS in a ratio of about 76.5%, 17.5%, 4.3%, and 1.7% respectively may also be used as the peptide cleaving and global deprotecting reagent.

The solvent used in step (4) of the process of the present invention may be selected from but not limited to dichloromethane, trichloromethane or the like. Dichloromethane is used as preferred solvent.

The amount of TFA used for the purpose of cleavage of peptide from resin and global deprotection in the cocktail mixture may range from 60% to 90% with respect to its concentration in DCM. The temperature at which the cleavage and global deprotection is carried out may range from about 15° C. to about 40° C. Preferred temperature for cleavage and global deprotection may be in the range of about 25°-30° C. A process of global deprotection involves removal of protecting groups of all the multifunctional protected amino acids of the peptide sequence attached to the resin.

After the completion of the reaction, the reaction mixture may optionally be filtered and washed with acid or an organic solvent. Crude Bivalirudin may be isolated by combining the reaction mass with an organic solvent, preferably by combining with an ether solvent.

Ether solvents that may be used include but are not limited to diethyl ether, diisopropyl ether, tert-butyl methyl ether, tert-butyl ethyl ether, tert-amyl methyl ether, isopropyl ether and the like or combinations thereof.

The isolation may be carried out by adding the reaction mass is added to an ether solvent. More preferably, the reaction mass is added to an ether solvent precooled to a temperature of about −5° C. to about 5° C.

The obtained suspension may be maintained at a temperature of about 0° C. to about 15° C., preferably at a temperature of about 0° C. to about 5° C. to effect the complete precipitation of the product.

The obtained precipitate may be separated using conventional techniques known in the art. The obtained crude product may be optionally washed with an organic solvents preferably ether and subjected to drying under continuous nitrogen purging.

Step-6) Purifying Bivalirudin by Using a PLRP-S Column in Preparative HPLC

Step-6 of the process involves the purification of Bivalirudin on preparative HPLC, which comprises the steps of:
a) Purification by neutral gradient method on Preparative HPLC using PLRP-S column;
b) Purification by acid gradient method on Preparative HPLC using PLRP-S column; and
c) Isolating pure Bivalirudin.

Step-a): Purification by Neutral Gradient Method on Preparative HPLC Using PLRP-S Column.

Purification for crude Bivalirudin is carried out firstly in a neutral gradient medium, which comprises dissolution of crude peptide in ammonium acetate buffer and loading onto the column, which is packed with PLRP-S stationary phase. The stationary phase is a matrix of polystyrene-divinyl benzene co-polymer. The material is then eluted with a gradient of ammonium acetate (Buffer A) and methanol/acetonitrile (Buffer B) on a column.

Preferably the gradient of ammonium acetate (Buffer A) may have concentration of about 40 mM and methanol/acetonitrile (Buffer B) volumes may range from 50-75/50-25. During elution, fractions are collected at regular intervals using a PREP LC system. The collected fractions are assayed by HPLC to determine the purity and fraction with desired purities may be pooled together for further purification.

Step-b): Purification by Acid Gradient Method on Preparative HPLC Using PLRP-S Column.

The pooled fraction obtained from the previous purification step using neutral gradient is subjected to further purification separately using the same column by elution with a gradient comprising of buffer A: Orthophosphoric acid (adjusted to pH ˜3.0 with Triethylamine) and B: Acetonitrile.

In one of the preferred embodiment, the concentration of Orthophosphoric acid used as Buffer A may be about 0.3% to about 1% in water. During the elution, the desired pure fractions are collected again and assayed by HPLC.

Step-c) Isolating Pure Bivalirudin

The purified Bivalirudin pooled fraction from step-2) is then subjected to desolvatization to remove acetonitrile solvent. The desolvated concentrated pure pool is then loaded onto the same PLRP-S column for desalting step.

Desalting step is carried out by continuous washing of the loading column till the pH of the wash eluent increases to about ˜5.0. Preferably such washing with water may be repeated 3-4 times in order to get the complete salt removal.

Further, the column eluted with four volumes of 0.1% TFA in water followed by elution with a gradient of 0.1% TFA in water and acetonitrile to collect the TFA salt of Bivalirudin fractions and analyzed by HPLC assay method to ascertain the purity of fractions. Fractions with desired purity preferably greater than 96.5% purity may be considered as pure fractions.

The pure pooled fraction so obtained may be subjected to Lyophilization under the set parameters of Lyophilization to collect the lyophilized powder of Bivalirudin trifluoroacetate.

Bivalirudin obtained by the process of the present invention has the content of known impurities less than about 1.0%, preferably less than about 0.5% and the content of unknown impurities less than about 0.5% and preferably less than about 0.3%.

Further, moisture content by KF of Bivalirudin obtained by the process of the present invention may range from about 4% to 8% and preferably may range about 5% to 6%.

In another embodiment the present invention provides pharmaceutical compositions containing a therapeutically effective amount of Bivalirudin or pharmaceutically acceptable salts, its pharmaceutically acceptable analogs, polymorphs, solvates, or mixtures thereof, and processes for preparing the same. The pharmaceutical compositions of Bivalirudin optionally contain one or more pharmaceutically acceptable excipients.

In an embodiment of the present invention, the pharmaceutical composition comprising Bivalirudin or its pharmaceutically acceptable salt along with one or more pharmaceutically acceptable excipients may be formulated as: solid oral dosage forms including, but not limited to, powders, granules, pellets, tablets, capsules, liquid oral dosage forms including but not limited to syrups, suspensions, dispersions, and emulsions; and injectable compositions including but not limited to solutions, dispersions, and freeze dried compositions. The pharmaceutical composition of Bivalirudin may provide an immediate release, or modified release up on administration.

The term “Injectable composition” as used herein, includes compositions for any mode of administration that does not go through the digestive tract, but excludes trans-membrane delivery such as skin patches. Injectable composition most commonly refers to injections or infusions into blood vessels. In an embodiment, the mode of administration of injectable compositions of the present invention is by intravenous, intra-arterial, intrathecal, intraperitoneal, intratumoral, intra-articular, intramuscular or subcutaneous injection, and the like.

The injectable pharmaceutical compositions may optionally contain pharmaceutically acceptable additives such as preservatives, pH modifiers, buffering, chelating, complexing and solubilizing agents, antioxidants and antimicrobial preservatives, suspending and/or viscosity modifying agents, tonicity modifying agents, and other biocompatible materials or therapeutic agents.

Non-limiting examples of preservatives that can be employed in the context of present application include parabens such as methyl paraben and propyl paraben, meta-cresol, para-cresol, bronopol, benzalkonium chloride, and the like or mixtures thereof.

In another embodiment the present invention provides use of co-solvent or solubilizing agent in the compositions to solubilize other components of the system. Non-limiting examples of co-solvents, in the context of the present invention, include ethanol, propylene glycol, glycerol, glycofural, polyethylene glycol, diethylene glycol monoethyl ether (TRANSCUTOL®), polyethylene glycol 15 hydroxystearate (SOLUTOL®) and mixtures thereof.

The term “antioxidants” as used herein include metal ion chelators and/or reducing agents. A metal ion chelator functions as an antioxidant by binding to metal ions and thereby reduces the catalytic effect of metal ion on the oxidation reaction of the active and other components. Metal chelators that are useful include, but are not limited to, EDTA, glycine and citric acid or salts thereof. Non-limiting examples of antioxidants also include natural vitamin E, vitamin-E-succinate, ascorbic acid, sodium metabisulfite, amino acids, flavones, monothioglycerol, L-cysteine, thioglycolic acid and mixtures thereof. Such antioxidants may be used in the concentration range of 0.1 to 15% w/w, or 0.5 to 5% w/w.

Examples of pH modifiers and stabilizers include, but are not limited to, citric acid, tartaric acid, succinic acid, glutamic acid, ascorbic acid, lactic acid, acetic acid, malic acid, maleic acid, and sodium salts thereof, sodium hydroxide, sodium carbonate, sodium bicarbonate, tris buffer, meglumine, amino acids and mixtures thereof. Such pH modifiers and stabilizers maintain a desired pH between about 1.0 and 10.0 in the composition.

In an embodiment, the injectable composition of the present invention may be in the form of lyophilized products. Such lyophilized products may be diluted with aqueous fluid including water, various buffer solutions having different pH values, parenteral infusion fluids, and other such media before parenteral administration. Typically, parenteral infusion fluids include 5% dextrose solution, 0.9% sodium chloride solution, Ringer's lactate, mannitol infusion fluid, sucrose infusion fluid, plasma volume expanders, and mixtures thereof, at the time of parenteral administration.

The abbreviations used in the present description are defined below
Boc—tertiary-Butyloxycarbonyl
BOP—Benzotriazol-1-yl-oxy-tris-(dimethylamino)-phosphonium hexafluorophosphate
DBU—1,8-Diazobicycol[5.4.0]undec-7-ene
DCC—N,N-Dicyclohexylcarbodiimide
DIC—N,N-Diisopropylcarbodiimide
DCM—Dichloromethane
DMF—N,N-Dimethylformamide
EDT—1,2-Ethanedithiol
EDTA—Ethylenediamine tetra acetic acid
EEDQ—2-Ethoxy-1-ethoxycarbonyl-1,2-dihydroquinoline
DDM—Dodecane mercaptan
IIDQ—Isobutyl 1,2-dihydro-2-isobutoxy-1-quinolinecarboxylate
Fmoc—9-Fluorenylmethyloxycarbonyl
HBTU—2-(1H-Benzotriazol-1-yl)-1,1,3,3-tetramethyluronium hexafluorophosphate
HOBt—N-hydroxybenzotriazole
Pbf—2,2,4,6,7-Pentamethyldihydrobenzofuran-5-sulfonyl
PyBOP—Benzotriazol-1-yl-oxy-tris-(pyrrolidino)-phosphonium hexafluorophosphate
TBTU—O-Benzotriazol-1-yl-1,1,3,3-tetramethyluronium tetrafluoroborate
tBu—Tertiary butyl
TFA—Trifluoroacetic acid
TES—Triethylsilane
TIS—Triisopropylsilane
Trt—Trityl

The following examples are for illustration purposes only and are not intended to limit the scope of this invention.

### Experimental

All Fmoc protected amino acids were purchased from Adv. Chem. Tech.

Resins were purchased from Matrix Innovations Inc. and Rapp Polymere GBH and Adv. Chem. Tech.

### EXAMPLE 1

### Preparation of D-phenylalanyl-L-prolyl-L-arginyl-L-prolyl-glycyl-glycyl-glycyl-glycyl-L-asparaginyl-glycyl-L-alpha-aspartyl-L-phenylalanyl -L-alpha-glutamyl-L-alpha-glutamyl-L-isoleucyl-L-prolyl-L-alpha-glutamyl-L-alpha-glutamyl-L-tyrosyl-L-leucine trifluoroacetate hydrate using Tentagel S PHB resin

Fmoc-leucine (510 mg, 1.44 mmol) was dissolved in dichloromethane (3 ml) and dry tetrahydrofuran (0.2 ml). 1-(2-mesitylene sulfonyl)-3-nitro-1H-1,2,4 triazole (427 mg, 1.44 mmol) and 1-methyl imidazole (72 μl, 0.9 mmol) was then added. The reaction mixture was added to pre-swelled Tentagel S PHB resin (1 g, 0.24 mmol/g) in DCM and stirred for about 90 minutes at about 25° C. The above described sequence was repeated one more time to maximize the coupling efficiency. The capping was carried out using acetic anhydride (1 ml) DCM (8 ml) and pyridine (8 ml). The resin was washed with dichloromethane and DMF. The Fmoc protecting group was removed by treatment with 20% piperidine in DMF. The resin was washed repeatedly with DMF, DCM and DMF. The next amino acid, Fmoc-Tyr(tBu)-OH (666 mg, 1.44 mmol) was then added. The coupling was carried out by addition of HOBt (196 mg, 1.44 mmol) and DIC (182 mg, 1.44 mmol) in DMF. The completion of the coupling was confirmed by a ninhydrin test. After washing the resin, the Fmoc protecting group was removed with 20% piperidine in DMF. These steps were repeated each time with the respective amino acid according to the peptide sequence. Trifunctional amino acids were side chain protected as follows: Arg(Pbf), Asn(Trt), Asp(OtBu), Glu(OtBu), Tyr(tBu).

Cleavage of the peptide from the resin with simultaneous deprotection of the protecting group was carried out by treatment with ‘reagent K’ (82.5% TFA/5% phenol/5% thioanisole/5% water/2.5% 1,2 ethanedithiol) at RT. The cleavage mixture was collected by filtration. The resin was washed with TFA and dichloromethane. The excess of TFA and dichloromethane was concentrated to a small volume under nitrogen and dichloromethane was added to the residue and evaporated. The residue was allowed to cool. To the cooled residue, chilled ether was added to precipitate the peptide. The precipitated peptide was centrifuged. The residue was then dissolved in water and lyophilized to obtain the peptide as a white powder.

### EXAMPLE 2

### Preparation of D-phenylalanyl-L-prolyl-L-arginyl-L-prolyl-glycyl-glycyl-glycyl-glycyl-L-asparaginyl-glycyl-L-alpha-aspartyl-L-phenylalanyl -L-alpha-glutamyl-L-alpha-glutamyl-L-isoleucyl-L-prolyl-L-alpha-glutamyl-L-alpha-glutamyl-L-tyrosyl-L-leucine trifluoroacetate hydrate using Tentagel S AC resin

Tentagel S Ac Fmoc-leucine resin (500 mg, 0.25 mmol) was swelled in dichloromethane (about 10 ml) for about 2 hrs followed by DMF (about 10 ml) for about 2 hrs. The Fmoc protecting group was removed by treatment with 20% piperidine in DMF. The resin was washed repeatedly with DMF, DCM and DMF. The next amino acid, Fmoc-Tyr(tBu) (345 mg, 0.25 mmol) was then added. The coupling was carried out by addition of HOBt (102 mg, 0.25 mmol) and DIC (95 mg, 0.25 mmol) in DMF. The completion of the coupling was confirmed by ninhydrin test. After washing of the resin, the Fmoc protecting group was removed with 20% piperidine in DMF. The resin was washed with DMF, DCM and DMF before the addition of the next amino acid. These steps were repeated each time with the successive amino acid according to the peptide sequence. Trifunctional amino acids were side chain protected as follows: Arg(Pbf), Asn(Trt), Asp(OtBu), Glu(OtBu), Tyr(tBu).

Cleavage of the peptide from the resin with simultaneous deprotection of the protecting group was carried out by treatment with ‘reagent K’ (82.5% TFA/5% phenol/5% thioanisole/5% water/2.5% 1,2 ethanedithiol) at RT. The cleavage mixture was collected by filtration. The resin was washed with TFA and dichloromethane. The excess of TFA and dichloromethane was concentrated to a small volume and dichloromethane was added to the residue and evaporated. The residue was allowed to cool. To the cooled residue, chilled anhydrous ether was added to precipitate the peptide. The precipitated peptide was centrifuged. The residue was then dissolved in water and lyophilized to obtain the peptide as a white powder.

### EXAMPLE 3

### Preparation of D-phenylalanyl-L-prolyl-L-arginyl-L-prolyl-glycyl-glycyl-glycyl-glycyl-L-asparaginyl-glycyl-L-alpha-aspartyl-L-phenylalanyl -L-alpha-glutamyl-L-alpha-glutamyl-L-isoleucyl-L-prolyl-L-alpha-glutamyl-L-alpha-glutamyl-L-tyrosyl-L-leucine trifluoroacetate hydrate using Tentagel S Ac resin by parallel synthesizer

Tentagel S Ac Fmoc-leucine resin (500 mg, 0.25 mmol) was used for the synthesis of the title peptide. Successive addition of remaining amino acids was carrying out using an excess equivalent of amino acids.

After anchoring the first amino acid to the resin and capping with acetic anhydride by the method described in the example 1. The following protocol (Table 1) was used for the synthesis using a Symphony Parallel Synthesizer

TABLE 1

| S. No. | Step | Time |
|---|---|---|
| 1 | Deprotection | 7.30 min × 2 |
| | 20% Piperidine/DMF | |
| 2 | Washing | 30 sec × 3 each |
| | DMF, DCM, DMF | |
| 3 | Coupling | 2 h × 1 |
| | Fmoc AA/BTU/ | |
| | NMM/DMF | |
| 4 | Washing | 30 sec × 3 each |
| | DMF, DCM, DMF | |

Cleavage of the peptide from the resin with simultaneous deprotection of the protecting group was carried out by treatment with ‘reagent K’ (82.5% TFA/5% phenol/5% thioanisole/5% water/2.5% 1,2 ethanedithiol) at RT. The cleavage mixture was collected by filtration. The resin was washed with and dichloromethane. The excess of TFA and dichloromethane was concentrated to a small volume and dichloromethane was added to the residue and evaporated. The residue was allowed to cool. To the cooled residue, chilled anhydrous ether was added to precipitate the peptide. The precipitated peptide was centrifuged. The residue was then dissolved in water and lyophilized to obtain the peptide as white powder.

### EXAMPLE 4

### Preparation of D-phenylalanyl-L-prolyl-L-arginyl-L-prolyl-glycyl-glycyl-glycyl-glycyl-L-asparaginyl-glycyl-L-alpha-aspartyl-L-phenylalanyl -L-alpha-glutamyl-L-alpha-glutamyl-L-isoleucyl-L-prolyl-L-alpha-glutamyl-L-alpha-glutamyl-L-tyrosyl-L-leucine trifluoroacetate hydrate using Wang Chem Matrix resin by parallel synthesizer

Chem Matrix Wang Fmoc-leucine resin (100 mg, 0.73 mmol) was used for the synthesis of the title peptide. Successive addition of remaining amino acids was carrying out using an excess equivalent of amino acids.

After anchoring the first amino acid to the resin and capping with acetic anhydride by the method described in the example 1. The following protocol (Table 2) was used for the synthesis using a Symphony Parallel Synthesizer.

TABLE 2

| S. No. | Step | Time |
|---|---|---|
| 1 | Deprotection | 7.30 min × 2 |
| | 20% Piperidine/DMF | |
| 2 | Washing | 30 sec × 3 each |
| | DMF, DCM, DMF | |
| 3 | Coupling | 2 h × 1 |
| | Fmoc AA/HBTU/ | |
| | NMM/DMF | |
| 4 | Washing | 30 sec × 3 each |
| | DMF, DCM, DMF | |

Cleavage of the peptide from the resin with simultaneous deprotection of the protecting group was carried out by treatment with ‘reagent (82.5% TFA/5% phenol/5% thioanisole/5% water/2.5% 1,2 ethanedithiol) at about RT. The cleavage mixture was collected by filtration. The resin was washed with TFA and dichloromethane. The excess of TFA and dichloromethane was concentrated to a small volume and dichloromethane was added to the residue and evaporated. The residue was allowed to cool. To the cooled residue, chilled anhydrous ether was added to precipitate the peptide. The precipitated peptide was centrifuged. The residue was then dissolved in water and lyophilized to obtain the peptide as a white powder.

### EXAMPLE 5

### Preparation of D-phenylalanyl-L-prolyl-L-arginyl-L-prolyl-glycyl-glycyl-glycyl-glycyl-L-asparaginyl-glycyl-L-alpha-aspartyl-L-phenylalanyl -L-alpha-glutamyl-L-alpha-glycyl-L-asparaginyl-glycyl-L-alpha-aspartyl-L-phenylalanyl glutamyl-L-isoleucyl-L-prolyl-L-alpha-glutamyl-L-alpha-glutamyl-L-tyrosyl-L-leucine trifluoroacetate hydrate using Wang Chem Matrix resin

Chem Matrix Wang Fmoc-leucine resin (200 mg, 0.73 mmol) was used for the synthesis of the title peptide. The Fmoc protecting group was removed by treatment with 20% piperidine in DMF. The resin was repeatedly washed with DMF, DCM and DMF. The next amino acid, Fmoc-Tyr(tBu) (403 mg) was then added. The coupling was carried out by the addition of HOBt (120 mg, 0.73 mmol) and DIC (110 mg, 0.73 mmol) in DMF. The completion of the coupling was confirmed by ninhydrin test. After washing of the resin, the Fmoc protecting group was removed with 20% piperidine in DMF. These steps were repeated each time with another amino acid according to peptide sequence. The Trifunctional amino acids were side chain protected as follows: Arg(Pbf), Asn(Trt), Asp(OtBu), Glu(OtBu).

Cleavage of the peptide from the resin with simultaneous deprotection of the protecting group was carried out by treatment with ‘reagent K’ (82.5% TFA/5% phenol/5% thioanisole/5% water/2.5% 1,2 ethanedithiol) at RT. The cleavage mixture was collected by filtration. The resin was washed with TFA and dichloromethane. The excess of TFA and dichloromethane was concentrated to a small volume and dichloromethane was added to the residue and evaporated. The residue was allowed to cool. To the cooled residue, chilled anhydrous ether was added to precipitate the peptide. The precipitated peptide was centrifuged. The residue was then dissolved in water and lyophilized to obtain the peptide.

### EXAMPLE 6

### Preparation of D-phenylalanyl-L-prolyl-L-arginyl-L-prolyl-glycyl-glycyl-glycyl-glycyl-L-asparaginyl-glycyl-L-alpha-aspartyl-L-phenylalanyl -L-alpha-glutamyl-L-alpha-glutamyl-L-isoleucyl-L-prolyl-L-alpha-glutamyl-L-alpha-glutamyl-L-tyrosyl-L-leucine trifluoroacetate using Wang Resin

### Stage-I: Preparation of Protected Bivalirudin Anchored to Wang Resin:

a) Preparation of Fmoc-Leu-Resin

Charged 500 g of Wang Resin (1.2 mmol/g) and 5 L of dichloromethane in a 3 neck round bottom flask with sinter, and stirred the mass for 2 hours. Filtered the dichloromethane, added 5 L of DMF to the resin and stirred for 2 hours. Filtered the DMF, and charged the solution of 1.27 Kg Fmoc-Leu-OH, 1.06 Kg MSNT, 2.5 L of dichloromethane, 100 ml of tetrahydrofuran and 179 ml of 1-Methyl imidazole to the resin and stirred for 2 hours. Filtered the solution and charged a solution of 1.27 Kg Fmoc-Leu-OH, 1.06 Kg MSNT, 2.5 L of dichloromethane, 100 ml of tetrahydrofuran and 179 ml of 1-Methyl imidazole to the resin obtained and stirred for 2 hours. Filtered the amino acid solution, added a capping solution (4.25 L) comprising acetic anhydride, pyridine, and dichloromethane in the ratio of 1:8:8, to the resin and stirred for 20 minutes. Filtered the capping solution and washed the resin with 5×5 L DMF to give Fmoc-Leu-Resin.

b) Preparation of Fmoc-Tyr(tBu)-Leu-Resin

Charged 4 L of 20% PIP in DMF to the resin and stirred the mass for 15 minutes and filtered the solution. Charged 4 L of 20% PIP in DMF to the resin obtained and stirred the mass for 15 minutes. Washed the resin with 5×5 L DMF.

Charged a solution of 551 g Fmoc-Tyr(tBu)-OH, 162 g HOBt and 187 ml of DIC in 2 L of DMF to the resin and stirred for 2 hours at 25-30° C. Filtered the amino acid solution and washed the resin with 5×5 L DMF.

c) Preparation of Fmoc-Glu(OtBu)-Tyr(tBu)-Leu-Resin

Charged 4 L of 20% PIP in DMF to the resin and stirred the mass for 15 minutes and filtered the solution. Charged 4 L of 20% PIP in DMF to the resin obtained and stirred the mass for 15 minutes. Washed the resin with 5×5 L DMF.

Charged a solution of 510 g Fmoc-Glu(OtBu)-OH, 162 g HOBt and 187 ml DIC in 2 L DMF to the resin and stirred for 2 hours at 25-30° C. Filtered the solution and washed the resin with 5×5 L DMF.

d) Preparation of Fmoc-Glu(OtBu)-Glu(OtBu)-Tyr(tBu)-Leu-Resin

Charged 4 L of 20% PIP in DMF to the resin and stirred the mass for 15 minutes and filtered the solution. Charged 4 L of 20% PIP in DMF to the resin obtained and stirred the mass for 15 minutes. Washed the resin with 5×5 L DMF.

Charged a solution of 510 g Fmoc-Glu(OtBu)-OH, 162 g HOBt and 187 ml DIC in 2 L DMF to the resin and stirred for 2 hours at 25-30° C. Filtered the solution and washed the resin with 5×5 L DMF.

e) Preparation of Fmoc-Pro-Glu(OtBu)-Glu(OtBu)-Tyr(tBu)-Leu-Resin

Charged 4 L of 20% PIP in DMF to the resin and stirred the mass for 15 minutes and filtered the solution. Charged 4 L of 20% PIP in DMF to the resin obtained and stirred the mass for 15 minutes. Washed the resin with 5×5 L DMF.

Charged a solution of 404 g Fmoc-Pro-OH, 162 g HOBt and 187 ml DIC in 2 L DMF to the resin and stirred for 2 hours at 25-30° C. Filtered the solution and washed the resin with 5×5 L DMF.

f) Preparation of Fmoc-Ile-Pro-Glu(OtBu)-Glu(OtBu)-Tyr(tBu)-Leu-Resin

Charged 4 L of 20% PIP in DMF to the resin and stirred the mass for 15 minutes and filtered the solution. Charged 4 L of 20% PIP in DMF to the resin obtained and stirred the mass for 15 minutes. Washed the resin with 5×5 L DMF.

Charged a solution of 424 g Fmoc-Ile-OH, 162 g HOBt, 187 ml DIC in 2 L DMF to the resin and stirred for 2 hours at 25-30° C. Filtered the solution and washed the resin with 5×5 L DMF.

g) Preparation of Fmoc-Glu(OtBu)-Ile-Pro-Glu(OtBu)-Glu(OtBu)-Tyr(tBu)-Leu-Resin

Charged 4 L of 20% PIP in DMF to the resin and stirred the mass for 15 minutes and filtered the solution. Charged 4 L of 20% PIP in DMF to the resin obtained and stirred the mass for 15 minutes. Washed the resin with 5×5 L DMF.

Charged a solution of 510 g Fmoc-Glu(OtBu)-OH, 162 g HOBt and 187 ml DIC in 2 L DMF to the resin and stirred for 2 hours at 25-30° C. Filtered the solution and washed the resin with 5×5 L DMF.

h) Preparation of Fmoc-Glu(OtBu)-Glu(OtBu)-Ile-Pro-Glu(OtBu)-Glu(OtBu)-Tyr(tBu)-Leu-Resin

Charged 4 L of 20% PIP in DMF to the resin and stirred the mass for 15 minutes and filtered the solution. Charged 4 L of 20% PIP in DMF to the resin obtained and stirred the mass for 15 minutes. Washed the resin with 5×5 L DMF.

Charged a solution of 510 g Fmoc-Glu(OtBu)-OH, 162 g HOBt and 187 ml DIC in 2 L DMF to the resin and stirred for 2 hours at 25-30° C. Filtered the solution and washed the resin with 5×5 L DMF.

i) Preparation of Fmoc-Phe-Glu(OtBu)-Glu(OtBu)-Ile-Pro-Glu(OtBu)-Glu(OtBu)-Tyr(tBu)-Leu-Resin

Charged 4 L of 20% PIP in DMF to the resin and stirred the mass for 15 minutes and filtered the solution. Charged 4 L of 20% PIP in DMF to the resin obtained and stirred the mass for 15 minutes. Washed the resin with 5×5 L DMF.

Charged a solution of 465 g Fmoc-Phe-OH, 162 g HOBt and 187 ml DIC in 2 L DMF to the resin and stirred for 2 hours at 25-30° C. Filtered the solution and washed the resin with 5×5 L DMF.

j) Preparation of Fmoc-Asp(OtBu)-Phe-Glu(OtBu)-Glu(OtBu)-Ile-Pro-Glu(OtBu)-Glu(OtBu)-Tyr(tBu)-Leu-Resin

Charged 2 L of DMF precooled to 7-9° C., to the resin and stirred. Charged 2 L of 40% PIP in DMF precooled to 7-9° C. to the resin under stirring and maintained for 15 minutes. The reaction mixture is filtered, and again charged 2 L of DMF (precooled to 7-9° C.) to the resin and stirred. Further, charged 2 L of 40% PIP in DMF, precooled to 7-9° C. to the resin under stirring and maintained for 15 minutes. The reaction mass is filtered and washed the resin with 5×5 L DMF.

Charged a solution of 493 g Fmoc-Asp(OtBu)-OH, 162 g HOBt, 187 ml DIC in 2 L DMF and stirred for 2 hours at 25-30° C.

Charged a solution of 246 g Fmoc-Asp(OtBu)-OH, 81 g HOBt, 93.4 ml DIC in 2 L DMF and stirred for 2 hours at 25-30° C. Filtered the solution and washed the resin with 5×5 L DMF.

k) Preparation of Fmoc-Gly-Asp(OtBu)-Phe-Glu(OtBu)-Glu(OtBu)-Ile-Pro-Glu(OtBu)-Glu(OtBu)-Tyr(tBu)-Leu-Resin

Charged 4 L of 20% PIP in DMF to the resin and stirred the mass for 15 minutes and filtered the solution. Charged 4 L of 20% PIP in DMF to the resin obtained and stirred the mass for 15 minutes. Washed the resin with 5×5 L DMF.

Charged a solution of 356 g Fmoc-Gly-OH, 162 g HOBt,187 ml DIC in 2 L DMF and stirred for 2 hours at 25-30° C.

Charged a solution of 178 g Fmoc-Gly-OH, 81 g HOBt, 93.4 ml DIC in 2 L DMF and stirred for 2 hours at 25-30° C. Filtered the solution and washed the resin with 5×5 L DMF.

l) Preparation of Fmoc-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin

Charged 4 L of 20% PIP in DMF to the resin, stirred the mass for 15 minutes and filtered the solution. Charged 4 L of 20% PIP in DMF to the resin obtained and stirred the mass for 15 minutes. Washed the resin with 5×5 L DMF.

Charged a solution of 716 g Fmoc-Asn(trt)-OH, 162 g HOBt, 187 ml DIC in 2 L DMF and stirred for 2 hours at 25-30° C. Filtered the solution and washed the resin with 5×5 L DMF.

m) Preparation of Fmoc-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin

Charged 4 L of 20% PIP in DMF to the resin, stirred the mass for 15 minutes and filtered the solution. Charged 4 L of 20% PIP in DMF to the resin obtained and stirred the mass for 15 minutes. Washed the resin with 5×5 L DMF.

Charged a solution of 356 g Fmoc-Gly-OH, 162 g HOBt, 187 ml DIC in 2 L DMF and stirred for 2 hours at 25-30° C. Filtered the solution and washed the resin with 5×5 L DMF.

n) Preparation of Fmoc-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin

Charged 4 L of 20% PIP in DMF to the resin, stirred the mass for 15 minutes and filtered the solution. Charged 4 L of 20% PIP in DMF to the resin obtained and stirred the mass for 15 minutes. Washed the resin with 5×5 L DMF.

Charged a solution of 356 g Fmoc-Gly-OH, 162 g HOBt and 187 ml DIC in 2 L DMF and stirred for 2 hours at 25-30° C. Filtered the solution and washed the resin with 5×5 L DMF.

o) Preparation of Fmoc-Gly-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin

Charged 4 L of 20% PIP in DMF to the resin, stirred the mass for 15 minutes and filtered the solution. Charged 4 L of 20% PIP in DMF to the resin obtained and stirred the mass for 15 minutes. Washed the resin with 5×5 L DMF.

Charged a solution of 356 g Fmoc-Gly-OH, 162 g HOBt and 187 ml DIC in 2 L DMF and stirred for 2 hours at 25-30° C. Filtered the solution and washed the resin with 5×5 L DMF.

p) Preparation of Fmoc-Gly-Gly-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin

Charged 4 L of 20% PIP in DMF to the resin, stirred the mass for 15 minutes and filtered the solution. Charged 4 L of 20% PIP in DMF to the resin obtained and stirred the mass for 15 minutes. Washed the resin with 5×5 L DMF.

Charged a solution of 356 g Fmoc-Gly-OH, 162 g HOBt and 187 ml DIC in 2 L DMF and stirred for 2 hours at 25-30° C. Filtered the solution and washed the resin with 5×5 L DMF.

q) Preparation of Fmoc-Pro-Gly-Gly-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu-Resin

Charged 4 L of 20% PIP in DMF to the resin, stirred the mass for 15 minutes and filtered the solution. Charged 4 L of 20% PIP in DMF to the resin obtained and stirred the mass for 15 minutes. Washed the resin with 5×5 L DMF.

Charged a solution of 404 g Fmoc-Pro-OH, 162 g HOBt, 187 ml DIC in 2 L DMF and stirred for 2 hours at 25-30° C. Filtered the solution and washed the resin with 5×5 L DMF.

r) Preparation of Fmoc-Arg(Pbf)-Pro-Gly-Gly-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu)-Leu -Resin

Charged 4 L of 20% PIP in DMF to the resin, stirred the mass for 15 minutes and filtered the solution. Charged 4 L of 20% PIP in DMF to the resin obtained and stirred the mass for 15 minutes. Washed the resin with 5×5 L DMF.

Charged a solution of 778 g Fmoc-Arg(Pbf)-OH, 162 g HOBt and 187 ml DIC in 3 L DMF and stirred for 2 hours at 25-30° C.

Charged a solution of 778 g Fmoc-Arg(Pbf)-OH, 162 g HOBt and 187 ml DIC in 3 L DMF to the resin and stirred for 2 hours at 25-30° C.

Charged a solution of 778 g Fmoc-Arg(Pbf)-OH, 162 g HOBt and 187 ml DIC in 3 L DMF to the resin and stirred for 1 hours at 25-30° C. Filtered the solution and washed the resin with 5×5 L DMF.

s) Preparation of Fmoc-Pro-Arg(Pbf)-Pro-Gly-Gly-Gly-Gly-Asn(trt)-Gly-Asp(OtBu)-Phe-Glu(OtBu)-Glu(OtBu)-Ile-Pro-Glu(OtBu)-Glu(OtBu)-Tyr(tBu) -Leu-Resin

Charged 4 L of 20% PIP in DMF to the resin, stirred the mass for 15 minutes and filtered the solution. Charged 4 L of 20% PIP in DMF to the resin obtained and stirred the mass for 15 minutes. Washed the resin with 5×5 L DMF.

Charged a solution of 404 g Fmoc-Pro-OH, 162 g HOBt, 187 ml DIC in 2 L DMF to the resin and stirred for 2 hours at 25-30° C.

Charged a solution of 404 g Fmoc-Pro-OH, 162 g HOBt, 187 ml DIC in 2 L DMF to the resin and stirred for 2 hours at 25-30° C. Filtered the solution and washed the resin with 5×5 L DMF.

t) Preparation of Boc-D-Phe-Pro-Arg(Pbf)-Pro-Gly-Gly-Gly-Gly-Asn(trt)-Gly-Asp(OtBu)-Phe-Glu(OtBu)-Glu(OtBu)-Ile-Pro-Glu(OtBu)-Glu(OtBu) -Tyr(tBu)-Leu-Resin

Charged 4 L of 20% PIP in DMF to the resin, stirred the mass for 15 minutes and filtered the solution. Charged 4 L of 20% PIP in DMF to the resin obtained and stirred the mass for 15 minutes. Washed the resin with 5×5 L DMF.

Charged a solution of 318 g of Boc-D-Phe-OH, 162 g HOBt, 187 ml DIC in 2 L DMF to the resin and stirred for 2 hours at 25-30° C.

Charged a solution of 318 g of Boc-D-Phe-OH, 162 g HOBt, 187 ml DIC in 2 L DMF to the resin and stirred for 2 hours at 25-30° C. Filtered the solution and washed the resin with 5×5 L DMF. Washed the resin with 2×5 L methanol, followed by 2×5 L methyl tert butyl ether. The obtained resin is dried under vacuum (of about 580 to 620 mm Hg).

Stage-II: Cleavage of Peptide from Resin along with Global Deprotection

Charged 200 g of resin with peptide (obtained in stage I) in a round-bottomed flask and allowed to swell in 500 ml DCM for 15 to 20 minutes under nitrogen at 25-30° C. Charged 2.3 L of cocktail mixture of TFA (1.76 L), phenol (400 ml), water (100 ml) and TIPS (40 ml) to the resin at 25-30° C. and the obtained reaction mixture is stirred to for 2.5 hours at 25-30° C. under nitrogen. Filtered the reaction mixture and washed the resin with 250 ml of TFA. Charged the obtained filtrate to 4 L of cold MTBE (precooled to a temperature of 0±5° C.) under stirring by not allowing the temperature to rise more than 5° C. Stirred the reaction mixture for 45-75 minutes at 0-5° C. and the obtained suspension is filtered, washed the solid with 5×1 L MTBE and dry the solid under nitrogen.

### Stage-III: Purification of Crude Bivalirudin Using PLRP-S Column in Preparative HPLC:

The purification of Bivalirudin is carried in two steps as follows:
1. Purification of crude by ammonium acetate based neutral gradient method
2. Purification of ammonium acetate purified fractions by o-Phosphoric acid based acid gradient method.

### 1. Purification of Crude by Ammonium Acetate Based Neutral Gradient Method:

The crude material cleaved from the resin is dissolved in 40 mM ammonium acetate buffer and loaded onto the column. The material is then eluted with a gradient of 40 mM ammonium acetate (Buffer A) and 75/25 methanol/acetonitrile (Buffer B) on a column packed with PLRP-S stationary phase. The stationary phase is a matrix of polystyrene-divinyl benzene co-polymer. During elution, fractions are collected at regular intervals using a PREP LC system. The fractions of desired purity are collected and assayed by HPLC to determine the purity. Fractions with significant amount of Bivalirudin are pooled and preceded for the next step of purification.

2. Purification of Ammonium Acetate Purified Fractions by o-Phosphoric Acid Based Acid Gradient Method.

Each pure pool obtained from the previous purification is further purified separately using the same column by elution with a gradient comprising of buffer A: 0.3% Orthophosphoric acid (adjusted to pH ˜3.0 with TEA) and B: 100% acetonitrile. During the elution, fractions are collected again and assayed by HPLC. Fractions that meet API specification of >96.5% Bivalirudin purity with NMT 1% known impurities and NMT 0.5% unknown impurities are pooled together for final desalting.

The prep purified Bivalirudin containing pure pool is then desolvatized to remove acetonitrile. The concentrated pure pool is then loaded onto the same PLRP-S column for final desalting. After loading, the column is washed with water till the pH of the wash eluent increases to ˜5.0. This is generally achieved in 3-4 column volume washing. After this, the column is washed with four volumes of 0.1% TFA in water. Once the washing is done, Bivalirudin is eluted as a TFA salt with a gradient of water+acetonitrile with 0.1% TFA. During this elution process, fractions are collected over a period of time and analyzed by HPLC assay method to ascertain the purity of fractions. Fractions with greater than 96.0% purity are collected and pooled. The obtained solution of Bivalirudin is lyophilized to give a Bivalirudin trifluoroacetate as a white powder.

Moisture content by KF: 5.41%

Purity by HPLC: >96.5%

1. An improved process for the preparation of Bivalirudin comprising the steps of:
1) Anchoring the first protected terminal amino acid to a resin using MSNT and 1-methyl imidazole;
2) Capping of resin obtained after step-1, using anhydride of acetic acid;
3) Selectively deprotecting the amino acid using nucleophilic base;
4) Coupling the carboxyl terminus of the next N-protected amino acid to the amine from step 3) in presence of DIC and HOBt;
5) Repeating steps 3) and 4) to synthesize the desired peptide sequence;
6) Cleaving the peptide from the resin and global deprotection using cocktail mixture comprising a composition of TFA/Phenol/Water/TIPS to obtain crude Bivalirudin; and
7) Purifying Crude Bivalirudin by using a PLRP-S column in preparative HPLC by neutral gradient followed by acid gradient method.

2. A process according to claim 1, wherein the resin used for the preparation of Bivalirudin is selected from TentaGel TGA, TentaGel S PHB, TentaGel S AC, ChemMatrix Wang, Wang resin (1.2 mmol/g) or HMPB Chem Matrix.

3. A process according to claim 1, wherein the resin used for the preparation of Bivalirudin is Wang resin possessing 1.2 m mol/gm of loading capacity.

4. A process according to claim 1, wherein the amount of protected amino acid used in the range from about 1 to about 7 molar equivalents, per molar equivalent of resin with respect to resin loading capacity.

5. A process according to claim 4, wherein the amount of protected amino acids used is an amount of about 2 to about 4 molar equivalent per molar equivalent of resin with resin loading capacity.

6. A process according to claim 5, wherein the amount of protected amino acids used is an amount of about 1.5 to about 2.5 molar equivalent for amino acids selected from Tyr, Glu, Pro, Ileu, Phe, Asn, and Gly per molar equivalent of resin with respect to resin loading capacity.

7. A process according to claim 4, wherein amino acids used is an amount of about 5.5 to about 6.5 molar equivalent for amino acid Arg per molar equivalent of resin with respect to resin loading capacity.

8. A process according to claim 1, wherein molar ratio of condensing agent MSNT and 1-methylimidazole is about 1M to 8M per molar equivalent of resin with respect to resin loading capacity independently.

9. A process according to claim 1, wherein capping solution of step-2 comprises of acetic anhydride, pyridine, and dichloromethane in the ratio of about 1:8:8.

10. A process according to claim 1, wherein nucleophilic base is piperidine dissolved in DMF solvent.

11. A process according to claim 10, wherein piperidine dissolved in DMF solvent is ranging from about 15% to about 50% v/v.

12. A process according to claim 1, wherein the molar ratio of condensing agent DIC and HOBt is about 1M to 6M per molar equivalent of resin with respect to resin loading capacity independently.

13. A process according to claim 1, wherein the cocktail mixture comprises a composition of TFA/Phenol/Water/TIPS in the preferred proportions of about 76.5%/17.5%/4.3% /1.7% respectively.

14. A process according to claims 1, wherein the step-6 is preferably carried out in dichloromethane.

15. A process for the purification of Bivalirudin, which comprises the steps of:
1) Purification by neutral gradient method on Preparative HPLC using PLRP-S column
2) Purification by acid gradient method on Preparative HPLC using PLRP-S column
3) Isolating pure Bivalirudin.

16. A process according to claim 15, wherein step-1) comprising neutral gradient method involves gradient elution with Buffer-A prepared from ammonium acetate and Buffer-B prepared from methanol and acetonitrile.

17. A process according to claim 16, wherein step-2) comprising acid gradient method involves gradient elution with Buffer-A prepared from orthophosphoric acid and triethyl amine, and Buffer-B of acetonitrile.

18. A process according to claim 16, wherein step-3) comprising isolating pure Bivalirudin involves desolvation and desalting on Preparative HPLC using PLRP-S column.

19. A process according to claim 18 further comprises salt formation using 0.1% TFA in acetonitrile and water followed by Lyophilization.

20. Pure Bivalirudin obtained after the purification according to claim 15, having known impurities contents less than about 1%.

21. Pure Bivalirudin obtained after the purification according to claim 15, having unknown impurities contents less than about 0.5%.

22. Pure Bivalirudin according to claim 21 wherein the unknown impurities have RRT valves selected from about 0.601, 0.894, 0.934, 1.090 and 1.096.
